(19) United States
(12) Patent Application Publication
(10) Pub. No.: US 2014/0309659 A1
(43) Pub. Date: Oct. 16, 2014

(54) SURGICAL ROBOT AND CONTROL METHOD THEREOF

(71) Applicant: SAMSUNG ELECTRONICS CO., LTD., Suwon-si (KR)

(72) Inventors: Se Gon ROH, Suwon-si (KR); Young Do Kwon, Yongin-si (KR); Woong Kwon, Seongnam-si (KR); Yong Jae Kim, Seoul (KR); Jeong Hun Kim, Hwaseong-si (KR); Kyung Shik Roh, Seongnam-si (KR); Youn Baek Lee, Suwon-si (KR); Jong Won Lee, Uiwang-City (KR); Byung June Choi, Gunpo-si (KR)

(73) Assignee: SAMSUNG ELECTRONICS CO., LTD., Suwon-si (KR)

(21) Appl. No.: 14/192,186

(22) Filed: Feb. 27, 2014

(30) Foreign Application Priority Data
Apr. 10, 2013 (KR) 10-2013-0039098

(51) Int. Cl. A61B 19/00

(52) U.S. Cl. CPC A61B 19/2203
USPC 606/130

(57) ABSTRACT

A surgical robot including a slave arm and an instrument provided at the slave arm to be introduced into a single port to perform surgery. The instrument includes a plurality of surgical instrument members to perform surgery while coming into contact with a surgical object, and a plurality of arm members. The arm members include surgical position regulators to move the surgical instrument members from the single port to a first surgical region where the surgical object is located, and surgical workers connecting the surgical position regulators and the surgical instrument members to each other, the surgical workers serving to move the surgical instrument members to a position close to surgical object within the first surgical region. The single-port surgical robot may effectively perform simultaneous surgery upon various surgical regions like multi-port surgery.

### CROSS-REFERENCE TO RELATED APPLICATIONS

This application claims the benefit of Korean Patent Applications No. 10-2013-0039098, filed on Apr. 10, 2013 in the Korean Intellectual Property Office, the disclosure of which is incorporated herein by reference.

### BACKGROUND

1. Field

Embodiments of the present invention relate to a surgical robot having an improved configuration to expand a surgical workspace and a control method thereof,

2. Description of the Related Art

Minimally invasive surgery refers to a surgical technique to minimize the size of an incision for surgery by inserting surgical instruments through several small incisions. Such minimally invasive surgery may reduce change in the metabolism of a patient after surgery, which is helpful to early recovery of the patient. Accordingly, minimally invasive surgery may shorten hospitalization after surgery as well as rapid return to normal life. In addition, minimally invasive surgery may achieve low pain after surgery and superior cosmetic effects owing to small incision size.

The most general form of minimally invasive surgery is endoscopic surgery. The most common form of endoscopic surgery is laparoscopic surgery that implements surgical investigation and minimally invasive surgery within the abdominal cavity. Upon implementation of standard laparoscopic surgery, after a small incision (about ½ inch or less) is formed to provide an entrance for laparoscopic surgical instruments in a state in which the patient's abdomen is filled with gas, a trocar is inserted through the incision. The laparoscope surgical instruments generally include a laparoscope (for observation of a surgical site) and other work instruments. Here, the work instruments are similar to those used in conventional open surgery, except that an operating end of each instrument or a distal operating piece is spaced apart from a handle by a shaft. Examples of the work instruments may include a clamp, a grasper, scissors, a stapler, and a needle holder. To implement surgery, a user inserts work instruments into a surgical site via the trocar and manipulates the instruments at the outside of the abdominal cavity.

The user monitors the progress of surgery via a monitor that displays an image of a surgical site captured by a laparoscope. Similar endoscopic technologies are applied to a retroperitoneoscope, pelviscope, arthroscope, sinoscope, hysteroscope, ureterorenoscope, cystoscope, urethroscope, pyeloscope, and the like.

However, in the case of multi-port surgery as one form of minimally invasive surgery that adopts a plurality of incisions formed in a part of the patient's body and a plurality of slave arms, although this enables surgery over a relatively wide area, the plurality of incisions may leave scars or extended recovery time may be required.

On the other hand, single-port surgery using a single incision formed in a part of the patient's body may achieve only a narrow surgical area, although it may minimize scarring and ensure early recovery.

### SUMMARY

It is one aspect of the present invention to provide a surgical robot that may provide an expanded surgical area upon single-port surgery and a control method thereof.

It is another aspect of the present invention to provide a surgical robot that enables surgery of organs distant from an incision owing to an organ avoidance path generation configuration and a control method thereof.

Additional aspects of the invention will be set forth in part in the description which follows and, in part, will be obvious from the description, or may be learned by practice of the invention.

In accordance with one aspect of the invention, a surgical robot includes a slave, and an instrument provided at the slave arm, the instrument being configured to be introduced into a single port incised in the patient's body to perform surgical motion, wherein the instrument includes a plurality of surgical instrument members configured to perform surgical work while coming into contact with at least one organ that is a surgical object, and a plurality of arm members, wherein the arm members include a plurality of surgical position regulators configured to move the plurality of surgical instrument members from the single to a first surgical region where the organ is placed, and a plurality of surgical workers connecting the surgical position regulators and the plurality of surgical instrument members to each other, the plurality of surgical workers serving to move the plurality of surgical instrument members to a position close to the organ within the first surgical region.

The slave arm may have an actuator.

The surgical position regulators may include shoulder joints configured to distribute the plurality of arm members in different directions or to gather the plurality of arm members in a given direction, and shoulder links coupled to the shoulder joints and configured to support the surgical workers.

The surgical position regulators may include a plurality of shoulder links by which the surgical workers are supported, and the plurality of shoulder links may be radially distributed, or be gathered in a first direction toward at least one surgical region among the plurality of surgical regions.

The shoulder joints may have at least 1 degree of freedom in a pitch direction or in a roll direction.

The shoulder links may have variable lengths to move the surgical workers to the first surgical region.

The shoulder links may include a first shoulder link provided toward the slave arm, and a second shoulder link provided toward the surgical worker, and the shoulder joints may include a first shoulder joint provided at the end of the slave arm, the first shoulder joint serving to determine orientation of the first shoulder link thereabout, and a second shoulder joint coupled to the first shoulder link, the second shoulder joint serving to determine orientation of the second shoulder link thereabout.

Each of the surgical workers may include an elbow link, one end of which is provided at an end of the surgical position regulator, an elbow joint to determine orientation of the elbow link at the end of the surgical position regulator, and a wrist type joint to determine orientation of the surgical instrument member at the other end of the elbow link.

The elbow link may have a variable length to allow the surgical instrument member to perform surgery upon the organ in the first surgical region.

The elbow joint may have at least 2 degrees of freedom in a pitch direction, in a roll direction, or in a yaw direction.

The surgical position regulators may be fixed after the surgical workers are moved to the first surgical region.

The plurality of arm members may include two main arms configured to perform surgical work, and one auxiliary arm configured to assist surgical work of the main arms.

The instrument may further include at least one camera arm, to which a camera to transmit information on a surgical state inside the patient's body is mounted.

In accordance with another aspect of the invention, a surgical robot includes a slave arm having an actuator, and an instrument provided at the slave arm, the instrument being configured to be introduced into a single port incised in the patient's body to perform surgical motion, wherein the instrument includes a plurality of surgical instrument members configured to perform surgical work while coming into contact with at least one organ that is a surgical object, and a plurality of arm members introduced into the patient's body, the plurality of arm members being configured to connect the plurality of surgical instrument members and the slave arm to each other, and wherein the plurality of arm members includes shoulder joints provided at an end of the slave arm, the shoulder joints having at least 2 degrees of freedom to radially distribute the plurality of surgical instrument members to a plurality of surgical regions where a plurality of surgical sites spaced apart from one another is located, or to gather the plurality of surgical instrument members in a given direction, and shoulder links coupled to the shoulder joints, lengths of the respective shoulder links being variable such that the shoulder links extend to the plurality of surgical regions located at different distances from the single port.

Each of the plurality of arm members may include an elbow link, one end of which is connected to one end of the shoulder link, and the other end of which is connected to the surgical instrument member, an elbow joint having at least 2 degrees of freedom to allow the elbow link to perform motion at the end of the shoulder link, and a wrist type joint having at least 2 degrees of freedom to allow the surgical instrument member to perform motion at the other end of the elbow link.

The shoulder links may have a variable length.

The plurality of arm members may include a camera arm equipped at an end thereof with a camera.

In accordance with another aspect of the invention, a surgical robot includes a slave arm having an actuator, and an instrument provided at the slave arm, the instrument being configured to be introduced into the patient's body, wherein the instrument includes a plurality of surgical instrument members configured to perform surgical work while coming into contact with at least one organ, and a plurality of arm members having shoulder joints provided at an end of the slave arm, wherein the shoulder joints have at least 2 degrees of freedom to distribute the plurality of arm members in different directions or to gather the plurality of arm members in a given direction.

In accordance with a further aspect of the invention, a control method of a surgical robot, the surgical robot including a slave arm having an actuator, a plurality of surgical instrument members provided at the slave arm and configured to be introduced into a single port incised in the patient's body to perform surgery, surgical position regulators to move the plurality of surgical instrument members to a surgical region, and surgical workers to move the plurality of surgical instrument members to an organ that is a surgical object placed in the surgical region, includes introducing the plurality of surgical instrument members into the patient's body through the single port, operating the surgical position regulators to move the plurality of surgical instrument members to a first surgical region where the organ that is a surgical object spaced apart from the single port is located, operating the surgical workers to move the plurality of surgical instrument members to a position close to the organ that is a surgical object within the first surgical region, and performing surgery using the plurality of surgical instrument members.

The surgical position regulators may be fixed after the plurality of surgical instrument members is moved to the first surgical region.

### BRIEF DESCRIPTION OF THE DRAWINGS

These and/or other aspects of the invention will become apparent and more readily appreciated from the following description of the embodiments, taken in conjunction with the accompanying drawings of which:

FIG. 1 is a view showing a surgical robot according to one embodiment of the present invention;

FIG. 2 is a view showing an instrument according to one embodiment of the present invention;

FIGS. 3A, 3B and 3C are views showing motion of the instrument of FIG. 2;

FIG. 4 is a view showing an instrument according to another embodiment of the present invention;

FIG. 5 is a view showing motion of the instrument of FIG. 4;

FIGS. 6A and 6B are views showing motion of a multi-port surgical robot and motion of a single-port surgical robot with respect to the same surgical range; and

FIGS. 7A and 7B are flowcharts of motion of the instrument according to one embodiment of the present invention.

### DETAILED DESCRIPTION

Reference will now be made in detail to the embodiments, examples of which are illustrated in the accompanying drawings, wherein like reference numerals refer to the like elements throughout. The embodiments are described below to explain the present invention by referring to the figures.

Hereinafter, reference will now be made in detail to the embodiments of the present invention, examples of which are illustrated in the accompanying drawings, wherein like reference numerals refer to like elements throughout.

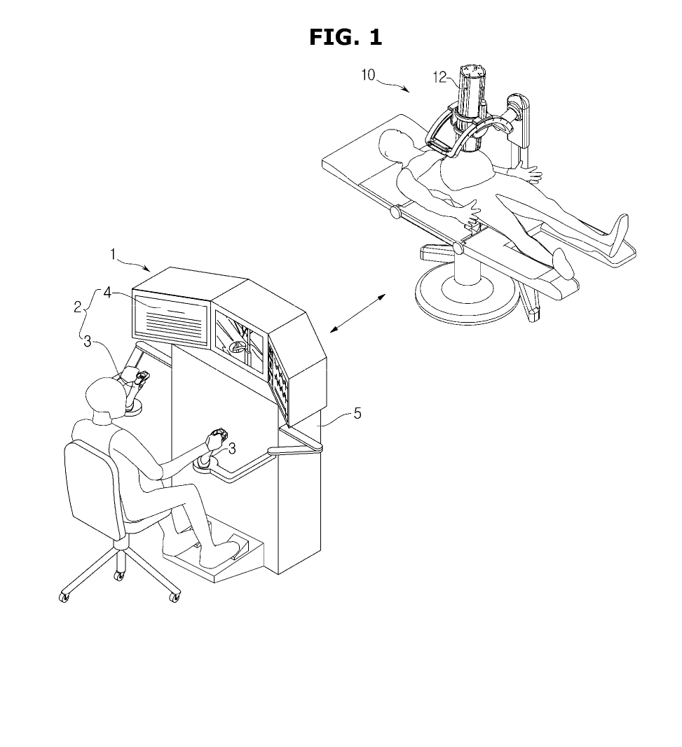

FIG. 1 is a view showing a surgical robot according to one embodiment of the present invention.

The surgical robot according to the present embodiment performs surgery by inserting an instrument into the body of a patient.

Referring to FIG. 1, the surgical robot includes a slave robot 10 to perform surgery on a patient who lies on an operating table, and a master console 1 to assist an operator in remotely controlling the slave robot 10. The master console 1 and the slave robot 10 may not be essentially physically separated from each other, and may constitute an integrated robot. In this case, a master interface 2, for example, may correspond to an interface part of the integrated robot.

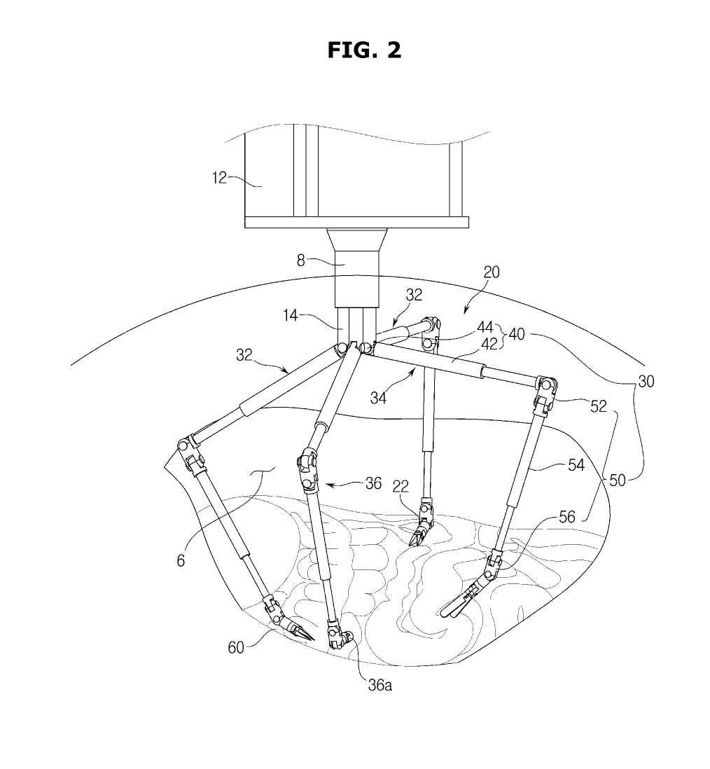

The master interface 2 of the master console 1 includes a monitor 4 and master controllers 5, and the slave robot 10 includes a slave arm 12 and an instrument 20 referring to FIG. 2. The instrument 20 is a surgical instrument, such as an endoscope including a celioscope, or a surgical operational member to directly manipulate a defective region, or the like. The following description is centered on the case in which the surgical instrument 20 is inserted into the patient's body 6 through a single port.

The master interface 2 includes the master controllers 5 that the operator will grip by both hands for manipulation. The master controllers, as exemplarily shown in FIG. 1, may be two handles 3. A manipulation signal generated as the operator manipulates the handles 3 is transmitted to the slave robot 10 to thereby control the slave arm 12. The operator may perform, e.g., position movement, rotation and cutting using the slave arm 12 and/or the instrument 20 by manipulating the handles 3.

For example, the handles 3 may include a main handle and a sub handle. Only one handle may be used to manipulate the slave arm 12 or the instrument 20, or the sub handle may be added to enable real-time simultaneous manipulation of a plurality of surgical equipment. The main handle and the sub handle may have various mechanical configurations depending on manipulation methods. For example, various input devices, such as a joystick, keypad, track-ball, touchscreen, etc., may be used to actuate the slave arm 12 of the slave robot 10 and/or other surgical equipment.

The master controller is not limited to the handle 3, and any other devices configured to control motion of the slave arm 12 via a network may be applied without any restrictions. The instrument 20 is mounted to a tip end of the surgical slave arm 12 having an actuator. As such, the instrument 20 is actuated upon receiving drive power from the actuator of the slave robot 10, thereby performing surgical motion. However, it is understood that the actuator may be installed in the instrument 20.

The monitor 4 of the master interface 2 displays an image input by, for example, a camera provided at the instrument 20. In addition, the monitor 4 may display various information depending on the kind of a selected image.

The slave robot 10 and the master console 1 may be coupled to each other via a wired communication network or a wireless communication network for transmission of, for example, a manipulation signal via the master interface 2 and an endoscopic image input via the instrument 20. If it is necessary to transmit two manipulation signals generated by the two handles 3 of the master interface 2 and/or a manipulation signal for position adjustment of the instrument 20 at the same time and/or similar times, the manipulation signals may be independently transmitted to the slave robot 10. Here, “independent” transmission of the respective manipulation signals refers to no interference between the manipulation signals and also refers to that any one manipulation signal has no effect on the other manipulation signal.

Accordingly, if the master console 1 generates a manipulation signal to control the surgical instrument 20 and a manipulation signal to control the slave robot 10, the respective manipulation signals may be independently transmitted to the slave robot 10 to drive an actuator (not shown) coupled to each device as described above.

To achieve independent transmission of the plurality of manipulation signals, various methods may be used. For example, header information may be added per manipulation signal upon generation of each manipulation signal, manipulation signals may be transmitted based on a generation sequence thereof, or manipulation signals may be transmitted based on a preset order of priority. In this case, it may be possible to fundamentally prevent interference between the respective manipulation signals by providing independent transmission paths of the respective signals.

Alternatively, one or more slave robots 10 may be used to perform surgery on the patient, the instrument 20 to display an image of a surgical site on the monitor 4 may serve as the independent slave robot 10, and the master console 1 may be integrated with the slave robot 10.

FIG. 2 is a view showing the instrument 20 according to one embodiment of the present invention.

The instrument 20 may be provided at an end of the slave arm 12 and may be introduced into an incised single port P3 of the patient to perform surgical motion.

Considering introduction of the instrument 20 through the single port P3 of the patient, a part of the patient's body 6 is incised for surgery, and carbon dioxide gas is introduced into an incision of the patient's body 6 to separate the internal organ inside the patient's body 6 from the skin.

A trocar 8 is inserted into the incision to facilitate introduction of the instrument 20.

The instrument 20 may include a plurality of surgical instrument members 22 that touch the organ B as a surgical site to perform surgical work, and a plurality of arm members 30 to control motion and positions of the plurality of surgical instrument members 22.

A main support arm 14 may be interposed between the slave arm 12 and the instrument 20 to assist the slave arm 12 in supporting the instrument 20. The main support arm 14 to assist the slave arm 12 in supporting the instrument 20 may be reciprocally movable or may have a variable length, to ensure introduction of the plurality of arm members 30 into the patient's body 6.

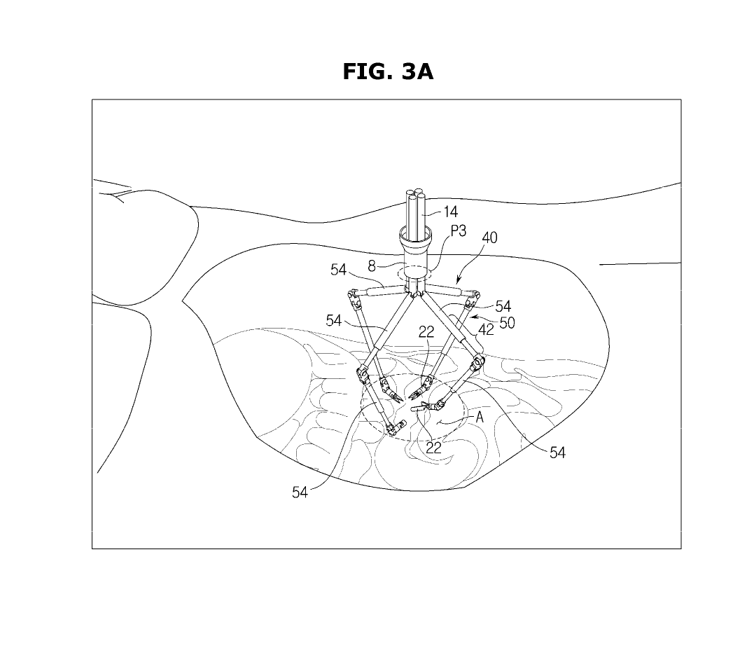

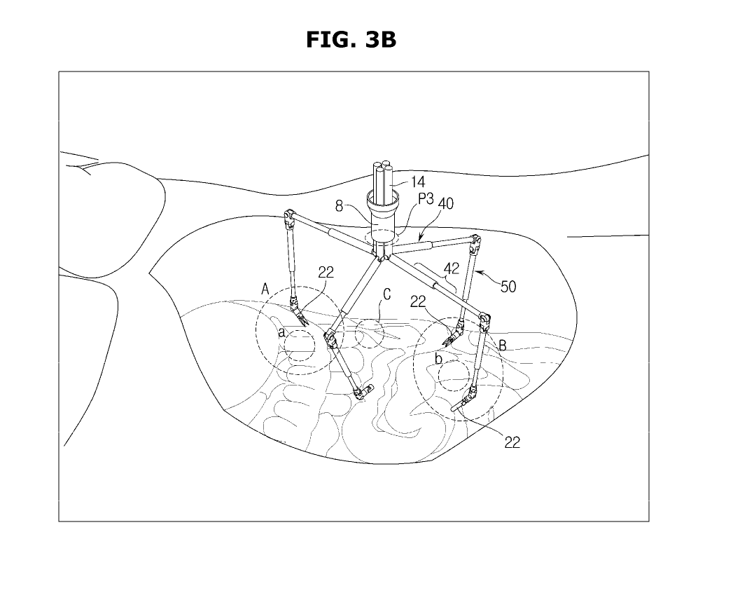

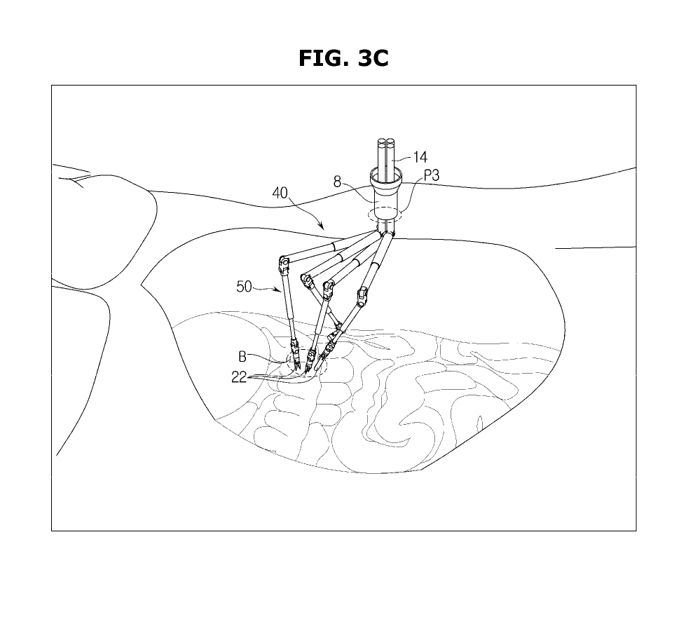

FIGS. 3A to 3C of the present embodiment, a plurality of surgical regions and a plurality of surgical sites spaced apart from one another may be simultaneously and/or sequentially treated. In the following description, for convenience of description, the plurality of surgical regions and the plurality of surgical sites include two surgical regions A and B and two surgical sites a and b.

The plurality of surgical instrument members 22 serves to contact the organ inside the patient's body 6, and may include a gripper, forceps, a jaw, and scissors, for example, to perform direct surgical motions, such as cutting and suturing. However, the present embodiment is not limited thereto, and any other surgical instrument members may be used.

The plurality of arm members 30 may be independently controlled.

The plurality of arm members 30 respectively includes surgical position regulators 40 to move the plurality of surgical instrument members 22 from a single port to the plurality of surgical regions A and B where the plurality of organs a and b is spaced apart from each other, and surgical workers 50 to connect the surgical position regulators 40 to the plurality of surgical instrument members 22, the surgical workers 50 serving to move the surgical instrument members 22 to positions close to the organ present in the corresponding surgical region.

The number of arm members 30 is not limited. In the present embodiment, for convenience of description, the plurality of arm members may include two main arms 32 to perform surgical work, such as cutting and suturing of the organ, and one auxiliary arm 34 to assist the surgical work of the main arms 32.

The lengths of the surgical position regulators 40 may vary to move the plurality of surgical instrument members 22 respectively to the plurality of surgical regions A and B (shown in FIGS. 3A-3D) spaced apart from each other.

The surgical position regulators 40 may include shoulder joints 44 provided at the end of the slave arm 12 to allow the plurality of arm members 30 to be distributed in different directions or to be gathered in a given direction, and shoulder links 42 coupled to the shoulder joints 44 to support the surgical workers 50. The shoulder joints 44 provided at the end of the slave arm 12 may determine the orientation of the shoulder links 42. More specifically, the shoulder joints 44 may be provided at one end of the main support arm 14 that is connected at the other end to the slave arm 12. In addition, the shoulder joints 44 may be provided at one end of the respective arm members 30, the other end of which is provided with the surgical instrument members 22. As such, the shoulder joints 44 may serve to determine the orientation of the plurality of arm members 30.

The shoulder joint 44 may have at least 1 degree of freedom to allow the shoulder link 42 to move in a pitch direction or in a roll direction about the main support arm 14. Referring to FIG. 2, the shoulder joint 44 may have 2 degrees of freedom for movement in both the pitch direction and the roll direction.

The pitch direction refers to a rotational direction about an axis of a horizontal plane that is orthogonal to a longitudinal direction, the roll direction refers to a rotational direction about an axis of a horizontal plane that is parallel to a longitudinal direction, and the yaw direction refers to a rotational direction about an axis of a vertical plane that is orthogonal to a longitudinal direction.

One end of the respective shoulder links 42 may be supported by the surgical workers 50 and the other end of the shoulder links 42 may be supported by the shoulder joints 44. The shoulder links 42 may have a variable length to move the surgical instrument members 22 from the incised single port of the patient's body to the organ located at the corresponding surgical region that is spaced apart from the single port.

The shoulder joints 44 and the shoulder links 42 included in the surgical position regulators 40 may serve to move the plurality of surgical workers 50 and the plurality of surgical instrument members 22 provided at the plurality of arm members 30 to a selected surgical region.

In the case of a multi-port surgical robot that requires a plurality of incisions formed in the patient's body, it may be advantageous to perform surgery over a wide region inside the patient's body 6 owing to provision of the plurality of incisions, but may suffer from long recovery time after surgery.

In the present embodiment, the plurality of arm members 30 inserted into the single port P3 may include the surgical position regulators 40, and the shoulder links 42 of the surgical position regulators 40 may be oriented in different directions. In addition, as exemplarily shown in FIGS. 3A and 3B, the lengths of the shoulder links 42 may be extended to enable surgery over a wider area. Accordingly, the single-port surgery may achieve superior effects equal to those of multi-port surgery. The surgical workers 50, which connect the surgical position regulators 40 and the plurality of surgical instrument members 22 to each other, function to move the plurality of surgical instrument members 22 to positions close to the organ of the corresponding surgical region.

The surgical workers 50 may respectively include elbow links 54 having one end connected to an end of the respective surgical position regulators 40, and elbow joints 52 connecting the surgical position regulators 40 and the elbow links 54 to each other. The surgical instrument members 22 may be provided at the other end of the elbow links 54.

The elbow links 54 are provided at the end of the surgical position regulators 40 and have a variable length. As such, after the plurality of surgical instrument members 22 is moved to, e.g., a first surgical region A by the surgical position regulators 40, the plurality of surgical instrument members 22 may be moved to positions close to the organ a as a surgical site by the elbow links 54.

The elbow joints 52, which connect the shoulder links 42 and the elbow links 54 to each other, may determine the orientation of the elbow links 54 at the end of the shoulder links 42. More specifically, the elbow joints 52 may be provided at one end of the shoulder links 42, the other end of which is connected to the main support arm 14. In addition, the elbow joints 52 may be provided at one end of the elbow links 54, the other end of which is provided with the surgical instrument members 22, and may serve to determine the orientation of the elbow links 54.

A wrist type joint 56 may be provided at the other end of the elbow link 54 to connect the elbow link 54 and the surgical instrument member 22 to each other, and may serve to determine the orientation of the surgical instrument member 22 at the end of the elbow link 54. More specifically, the wrist type joint 56 may be provided at the other end of the elbow link 54, one end of which is connected to the shoulder link 42.

The elbow joint 52 may allow the elbow link 54 to have at least 2 degrees of freedom in the pitch direction, the roll direction and/or the yaw direction about the shoulder link 42. Referring to FIG. 2, the elbow joint 52 may have 3 degrees of freedom in the pitch direction, the roll direction and the yaw direction.

The wrist type joint 56 may allow the surgical instrument member 22 to have at least 2 degrees of freedom in the pitch direction, the roll direction, and/or the yaw direction about the elbow link 54. Referring to FIG. 2, the wrist type joint 56 may have 3 degrees of freedom in the pitch direction, the roll direction and the yaw direction.

The instrument 20 may further include a camera arm 36, to which a camera 36a is mounted, to transmit information on a surgical state inside the patient's body to the master console 1.

FIGS. 3A to 3C are views showing movement of the instrument according to the present embodiment.

Specifically, FIGS. 3A to 3C show motion of the instrument 20 along an organ avoidance path to allow the instrument 20 to approach the surgical regions A and B while avoiding another organ c located between the surgical regions A and B.

In a state in which the plurality of shoulder links 42 of the plurality of arm members 30 is arranged in parallel, the lengths of the shoulder links 42 may be adjusted and the orientation of the shoulder links 42 may be adjusted via the plurality of shoulder joints 44. Thereafter, the elbow links 54 may be moved to move the surgical instrument members 22 to the surgical region A, and then the shoulder links 42 and the shoulder joints 44 may be fixed.

In this case, the shoulder links 42 and the shoulder joints 44 may be adjusted to allow the surgical instrument members 22 to perform surgical motion in the surgical region A while avoiding the organ c that is not a surgical object.

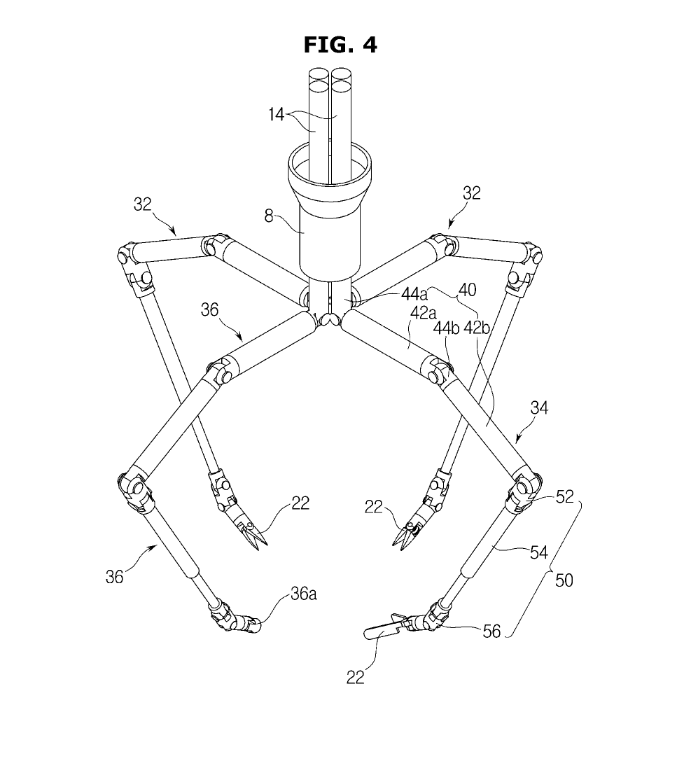

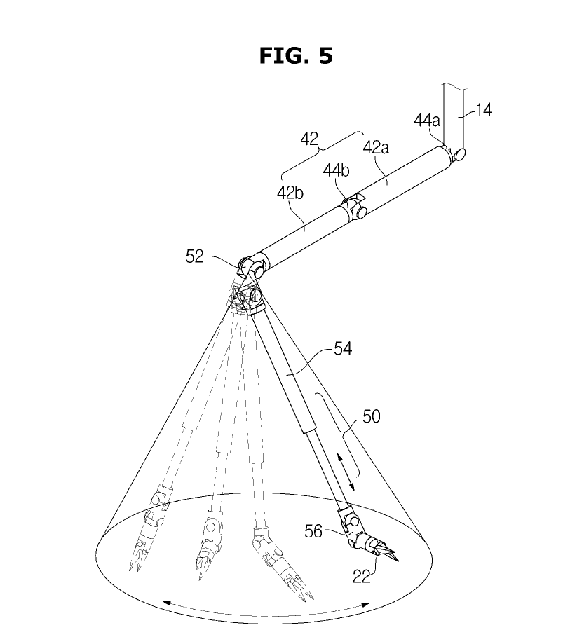

FIG. 4 is a view showing an instrument according to another embodiment of the present invention, and FIG. 5 is a view showing motion of the instrument of FIG. 4.

In the following description of the instrument 20 according to another embodiment of the present invention, a repeated description of the above-described instrument 20 will be omitted.

In the present embodiment, the plurality of shoulder links 42 is provided.

In the present embodiment, each of the shoulder links 42 may include a first shoulder link 42a and a second shoulder link 42b.

The first shoulder link 42a may be connected to the slave arm 12, more particularly to the main support arm 14. The second shoulder link 42b may be connected to the first shoulder link 42a and may support the surgical worker 50.

The first shoulder link 42a and the main support arm 14 are connected to each other via a first shoulder joint 44a, and the first shoulder link 42a and the second shoulder link 42b are connected to each other via a second shoulder joint 44b.

The first shoulder joint 44a may determine orientation of the first shoulder link 42a on the basis of the main support arm 14, and the second shoulder joint 44b may determine orientation of the second shoulder link 42b on the basis of the first shoulder link 42a.

The first shoulder joint 44a and the second shoulder joint 44b may have at least 2 degrees of freedom to move the first shoulder link 42a and the second shoulder link 42b in the pitch direction, the roll direction, and/or the yaw direction about the main support arm 14 and the first shoulder link 42a. FIG. 5 shows motion of the instrument 20. The motion of the instrument 20 achieved by the configuration of the present embodiment may be equally applied to the above-described embodiment.

After the first shoulder link 42a and the second shoulder link 42b of the shoulder link 42 are moved to the surgical region, the shoulder link 42 and the shoulder joints 44a and 44b may be fixed.

Thereafter, the elbow joint 52 of the surgical worker 50 may perform motion with at least 2 degrees of freedom, the length of the elbow link 54 may be adjusted, the wrist type joint 56 may perform motion with at least 2 degrees of freedom, and the surgical instrument member 22 may perform surgical motion on the corresponding organ. In this case, the surgical worker 50 may perform surgical motion within a conical workspace.

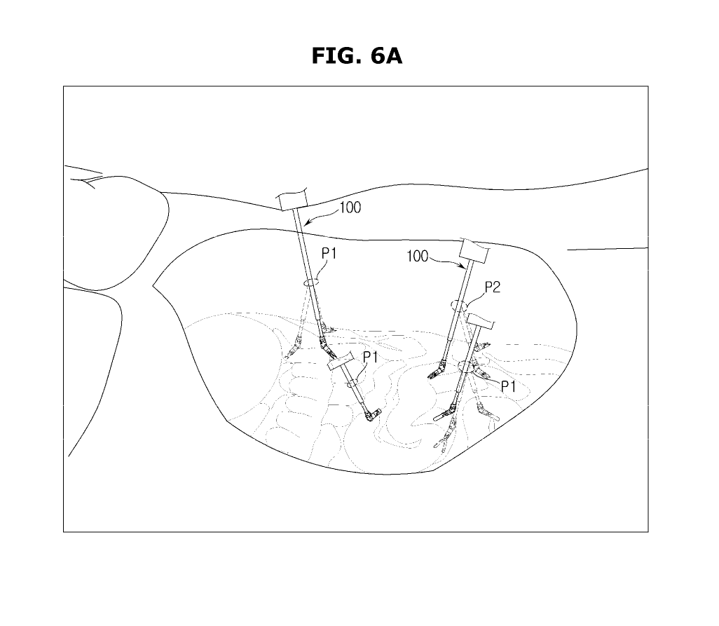

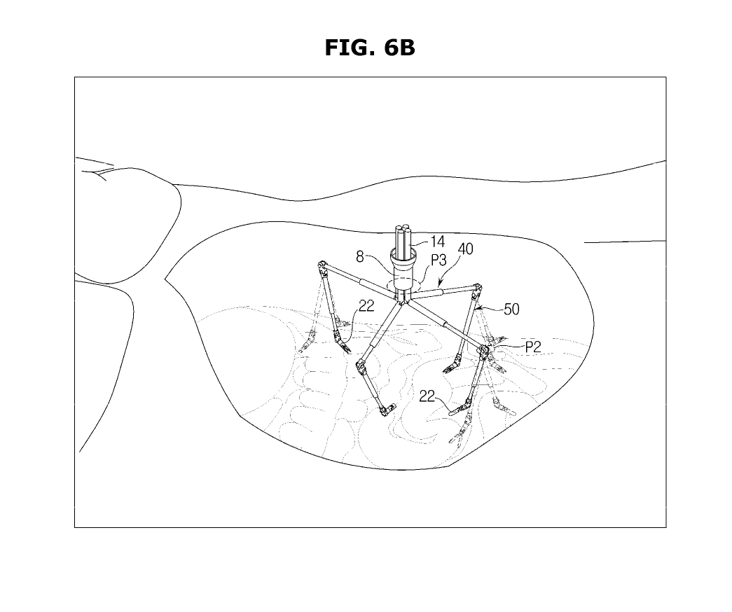

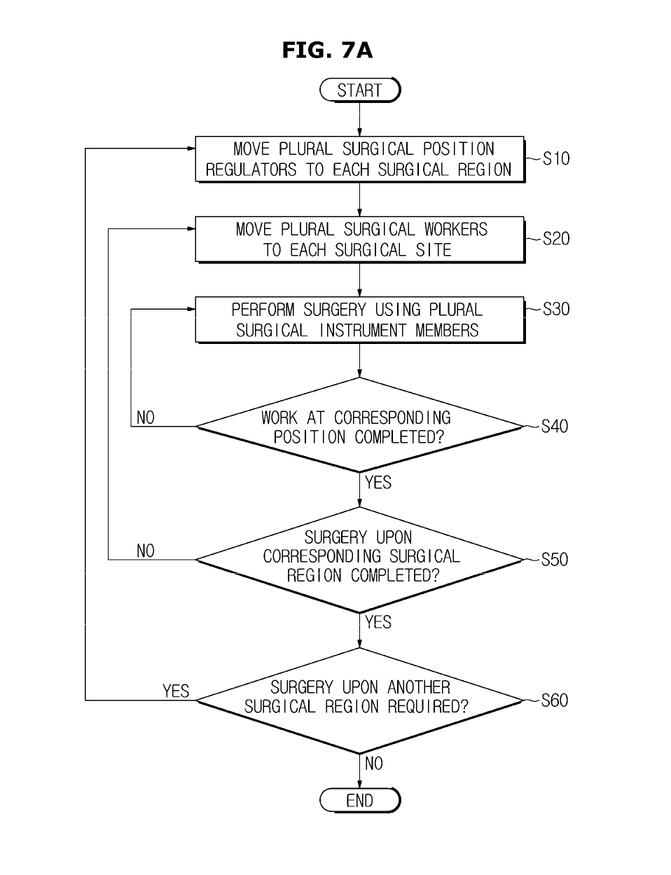

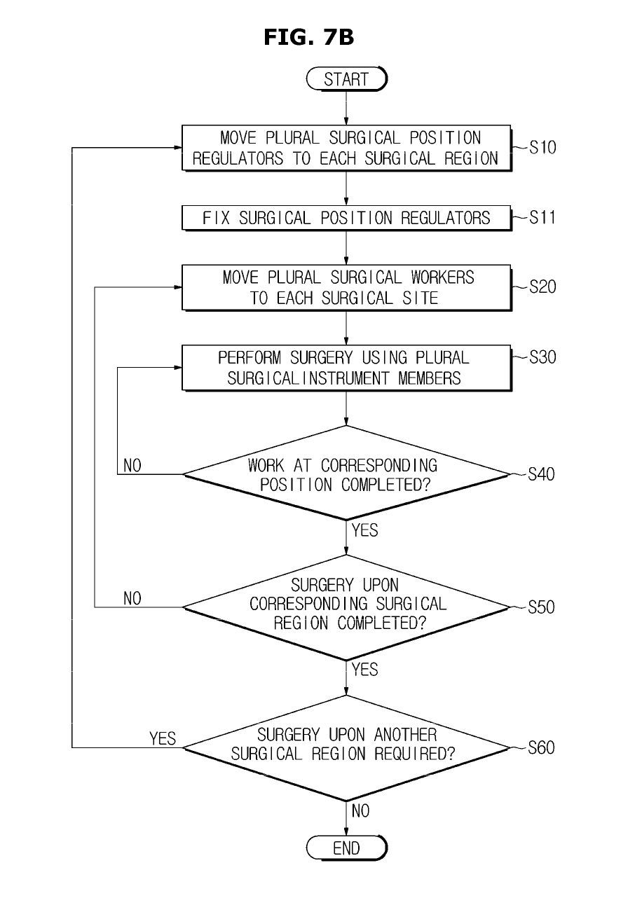

FIGS. 6A and 6B are views showing motion of a multi-port surgical robot and motion of a single-port surgical robot with respect to the same surgical range.

In the case of the multi-port surgical robot as exemplarily shown in FIG. 6A, a plurality of ports P1 and P2 is incised in the patient's body, and a plurality of robot arms 100 may be inserted into the ports P1 and P2 to perform surgical motion. In this case, each robot arm 100 may conically move about the port via length and orientation variation.

In the case of the single-port surgical robot as exemplarily shown in FIG. 6B, a single port P3 is incised in the patient's body, and the instrument 20 may be inserted into the single port P3 to perform surgical motion. In this case, the plurality of surgical position regulators 40 may proceed toward the plurality of surgical regions A and B respectively and may be fixed once the surgical regions A and B are reached.

In addition, the elbow joint 52 and the wrist type joint 56 of the surgical worker 50 may perform surgical work via conical movement about the elbow joint 52 and length variation.

With the above-described motion of the surgical position regulators 40 at the single port P3, despite provision of the single port, the single-port surgical robot may achieve the same operational effects as those of the multi-port surgical robot using the plurality of ports P1 and P2 as shown in FIG. 6A.

Hereinafter, a control method and motion of the surgical robot having the above-described configuration will be described.

The disclosure relates to a single-port surgical robot, the shape and setting of which may be changed according to a surgical work area to enable closer surgery, remote surgery, surgery over narrow and wide surgical areas, as well as organ avoidance surgery, like a multi-port surgical robot.

Once the patient lies on an operating table, the slave arm 12 is positioned over a surgical site of the patient by the master console 1.

To incise a port in the patient's body and insert the instrument 20 through the incised single port P3, the trocar 8 is inserted into the incised single port P3.

Thereafter, the instrument 20 provided at the end of the slave arm 12 is controlled to be inserted into the patient's body 6 through the trocar 8.

After insertion of the instrument 20, the orientation and length of each of the plurality of surgical position regulators 40 are adjusted to move the plurality of surgical instrument members 22 respectively to a plurality of surgical regions spaced apart from one another to enable implementation of surgical motion upon a plurality of surgical sites located in the surgical regions.

Since the plurality of surgical sites spaced apart from one another may have different orientations and lengths with respect to the incised single port P3, the lengths of the shoulder links 42 may be independently adjusted and orientation of the shoulder links 42 may be independently controlled via the shoulder joints 44. Thereby, the plurality of surgical instrument members 22 may be moved to the plurality of surgical regions A and B as exemplarily shown in FIG. 3B (S10).

In this case, if the plurality of surgical instrument members 22 reaches the plurality of surgical regions A and B, the plurality of surgical position regulators 40 may be fixed to minimize injury to the organs except for the surgical sites (S11).

Thereafter, as exemplarily shown in FIG. 3B, the surgical workers 50 may move the surgical instrument members 22 to positions close to the surgical sites a and b within the plurality of surgical regions A and B (S20), and cause the surgical instrument members 22 to perform surgery (S30).

If surgery by the surgical instrument members 22 at the surgical sites is completed (S40), whether or not surgery of the surgical sites is completed is judged (S50). The surgical workers 50 are moved to change positions of the surgical instrument members 22 until surgery of the surgical sites a and b is completed.

If surgery of the surgical sites a and b is completed and surgery of other sites outside the surgical regions A and B is required, the surgical position regulators 40 are released from a fixed state thereof and the surgical instrument members 22 are moved to second surgical regions where the corresponding surgical sites are located (S60).

While the surgical instrument members 22 are moved to the second surgical regions, the surgical position regulators 40 remain fixed, which may minimize injury to the organs located close thereto.

Thereafter, the surgical workers 50 move the surgical instrument members 22 to positions close to the corresponding surgical sites within the second surgical regions, and cause the surgical instrument members 22 to perform surgery. If surgery of all the surgical sites is completed via iteration of the above-described operations, the instrument 20 is retracted from the patient's body to complete surgery

As is apparent from the above description, a surgical robot and a control method thereof according to the embodiments of the present invention may enable surgery of a wide area using a single port as well as surgery of a surgical site distant from the single port without interference of other organs.

The apparatus and methods according to the above-described example embodiments may use one or more processors. For example, a processing device may be implemented using one or more general-purpose or special purpose computers, such as, for example, a processor, an image processor, a controller and an arithmetic logic unit, a central processing unit (CPU), a graphics processing unit (GPU), a digital signal processor (DSP), a microcomputer, a field programmable array, a programmable logic unit, an application-specific integrated circuit (ASIC), a microprocessor or any other device capable of responding to and executing instructions in a defined manner.

The terms “module”, and “unit,” as used herein, may refer to, but are not limited to, a software or hardware component or device, such as a Field Programmable Gate Array (FPGA) or Application Specific Integrated Circuit (ASIC), which performs certain tasks. A module or unit may be configured to reside on an addressable storage medium and configured to execute on one or more processors. Thus, a module or unit may include, by way of example, components, such as software components, object-oriented software components, class components and task components, processes, functions, attributes, procedures, subroutines, segments of program code, drivers, firmware, microcode, circuitry, data, databases, data structures, tables, arrays, and variables. The functionality provided for in the components and modules/units may be combined into fewer components and modules/units or further separated into additional components and modules.

Some example embodiments of the present disclosure can also be embodied as a computer readable medium including computer readable code/instruction to control at least one component of the above-described example embodiments. The medium may be any medium that can storage and/or transmission the computer readable code.

Aspects of the above-described example embodiments may be recorded in non-transitory computer-readable media including program instructions to implement various operations embodied by a computer. The media may also include, alone or in combination with the program instructions, data files, data structures, and the like. The program instructions recorded on the media may be those specially designed and constructed for the purposes of the example embodiments, or they may be of the kind well-known and available to those having skill in the computer software arts. Examples of non-transitory computer-readable media include magnetic media such as hard disks, floppy disks, and magnetic tape; optical media such as CD ROM disks and DVDs; magneto-optical media such as optical disks; and hardware devices that are specially configured to store and perform program instructions, such as read-only memory (ROM), random access memory (RAM), flash memory, and the like. The media may be transfer media such as optical lines, metal lines, or waveguides for transmitting a signal designating the program command and the data construction. Examples of program instructions include both machine code, such as produced by a compiler, and files containing higher level code that may be executed by the computer using an interpreter. The described hardware devices may be configured to act as one or more software modules in order to perform the operations of the above-described example embodiments, or vice versa. In addition, a non-transitory computer-readable storage medium may be distributed among computer systems connected through a network and computer-readable codes or program instructions may be stored and executed in a decentralized manner. In addition, the computer-readable storage media may also be embodied in at least one application specific integrated circuit (ASIC) or Field Programmable Gate Array (FPGA). Some or all of the operations performed according to the above-described example embodiments may be performed over a wired or wireless network, or a combination thereof.

Each block of the flowchart illustrations may represent a unit, module, segment, or portion of code, which comprises one or more executable instructions for implementing the specified logical function(s). It should also be noted that in some alternative implementations, the functions noted in the blocks may occur out of the order. For example, two blocks shown in succession may in fact be executed substantially concurrently or the blocks may sometimes be executed in the reverse order, depending upon the functionality involved. Also, while an illustration may show an example of the direction of flow of information for a process, the direction of flow of information may also be performed in the opposite direction for a same process or for a different process.

Although the embodiments of the present invention have been shown and described, it would be appreciated by those skilled in the art that changes may be made in these embodiments without departing from the principles and spirit of the invention, the scope of which is defined in the claims and their equivalents.

What is claimed is:

1. A surgical robot comprising:
a slave arm; and
an instrument provided at the slave arm, the instrument to be introduced into a single port to perform surgical motion,
wherein the instrument includes:
a plurality of surgical instrument members configured to perform surgical work while coming into contact with at least one organ that is a surgical object; and
a plurality of arm members introduced through the single port and coupled to the plurality of surgical instrument members, wherein the arm members include: a plurality of variable-length surgical position regulators to move the plurality of surgical instrument members respectively to a plurality of surgical regions where a plurality of surgical sites spaced apart from one another is located; and a plurality of surgical workers connecting the plurality of surgical position regulators and the plurality of surgical instrument members to each other, the plurality of surgical workers serving to move the plurality of surgical instrument members respectively to positions close to the plurality of surgical sites within the plurality of surgical regions.

2. The robot according to claim 1, wherein the plurality of arm members is controlled independently of each other.

3. The robot according to claim 1, wherein the plurality of surgical position regulators is fixed after ends of the surgical position regulators are respectively moved to positions above the plurality of surgical regions.

4. The robot according to claim 1, wherein the surgical position regulators include:
shoulder joints to distribute the plurality of arm members in different directions or to gather the plurality of arm members in a given direction; and
shoulder links coupled to the shoulder joints and to support the surgical workers.

5. The robot according to claim 4, wherein the shoulder links have variable lengths to move the surgical workers respectively to the plurality of surgical regions.

6. The robot according to claim 1, wherein the surgical position regulators include a plurality of shoulder links by which the surgical workers are supported, and
wherein the plurality of shoulder links is radially distributed, or is gathered in a first direction toward at least one surgical region among the plurality of surgical regions.

7. The robot according to claim 4, wherein the shoulder joints have at least 1 degree of freedom in a pitch direction or in a roll direction.

8. The robot according to claim 4, wherein the shoulder links include:
a first shoulder link provided toward the slave arm; and
a second shoulder link provided toward the surgical worker, and
wherein the shoulder joints include:
a first shoulder joint provided at the end of the slave arm, the first shoulder joint serving to determine orientation of the first shoulder link thereabout; and
a second shoulder joint coupled to the first shoulder link, the second shoulder joint serving to determine orientation of the second shoulder link thereabout.

9. The robot according to claim 1, wherein each of the surgical workers includes:
an elbow link, one end of which is provided at an end of the surgical position regulator;
an elbow joint to determine orientation of the elbow link at the end of the surgical position regulator; and
a wrist type joint to determine orientation of the surgical instrument member at the other end of the elbow link.

10. The robot according to claim 9, wherein the elbow link has a variable length to allow the surgical instrument member to perform surgery upon the organ in the first surgical region.

11. The robot according to claim 9, wherein the elbow joint has at least 2 degrees of freedom in a pitch direction, in a roll direction, or in a yaw direction.

12. The robot according to claim 1, wherein the plurality of arm members include:
two main arms to perform surgical work; and
one auxiliary arm to assist surgical work of the main arms.

13. The robot according to claim 11, wherein the instrument further includes at least one camera arm, to which a camera to transmit information on a surgical state inside the patient's body is mounted.

14. A surgical robot comprising:
a slave arm; and
an instrument provided at the slave arm, the instrument to be introduced into a single port to perform surgical motion,
wherein the instrument includes:
a plurality of surgical instrument members configured to perform surgical work while coming into contact with at least one organ that is a surgical object; and
a plurality of arm members introduced into the patient's body and coupled to the plurality of surgical instrument members, the plurality of arm members to connect the plurality of surgical instrument members and the slave arm to each other; wherein the plurality of arm members includes:
shoulder joints provided at an end of the slave arm, the shoulder joints having at least 2 degrees of freedom to radially distribute the plurality of surgical instrument members to a plurality of surgical regions where a plurality of surgical sites spaced apart from one another is located, or to gather the plurality of surgical instrument members in a given direction; and
shoulder links coupled to the shoulder joints, lengths of the respective shoulder links being variable such that the shoulder links extend to the plurality of surgical regions located at different distances from the single port.

15. The robot according to claim 14, wherein each of the plurality of arm members includes:
an elbow link, one end of which is connected to one end of the shoulder link, and the other end of which is connected to the surgical instrument member;
an elbow joint having at least 2 degrees of freedom to allow the elbow link to perform motion at the end of the shoulder link; and
a wrist type joint having at least 2 degrees of freedom to allow the surgical instrument member to perform motion at the other end of the elbow link.

16. The robot according to claim 14, wherein the shoulder links have a variable length.

17. A surgical robot comprising:
a slave arm; and
an instrument provided at the slave arm,
wherein the instrument includes:
a plurality of surgical instrument members configured to perform surgical work while coming into contact with at least one organ; and
a plurality of arm members, coupled to the plurality of surgical instrument members, having shoulder joints provided at an end of the slave arm, wherein the shoulder joints have at least 2 degrees of freedom to distribute the plurality of arm members in different directions or to gather the plurality of arm members in a given direction.

18. A surgical robot comprising:
a slave arm; and
an instrument provided at the slave arm, the instrument to be introduced into a single port to perform surgical motion,
wherein the instrument includes:
a plurality of surgical instrument members configured to perform surgical work while coming into contact with an organ that is a surgical object; and
a plurality of arm members coupled to the plurality of surgical instrument members, wherein the arm members include: a plurality of surgical position regulators to move the plurality of surgical instrument members from the single port to a first surgical region where a surgical object is placed; and a plurality of surgical workers connecting the surgical position regulators and the plurality of surgical instrument members to each other, the plurality of surgical workers serving to move the plurality of surgical instrument members to a position close to the organ within the first surgical region.

19. A control method of a surgical robot, the surgical robot comprising a slave arm, a plurality of surgical instrument members provided at the slave arm and to be introduced into a single port to perform surgery, surgical position regulators to move the plurality of surgical instrument members to a surgical region, and surgical workers to move the plurality of surgical instrument members to a surgical object placed in the surgical region, the method comprising:
introducing the plurality of surgical instrument members through the single port;
operating the surgical position regulators to move the plurality of surgical instrument members to a first surgical region where the surgical object spaced apart from the single port is located;
operating the surgical workers to move the plurality of surgical instrument members to a position close to the surgical object within the first surgical region; and
performing surgery using the plurality of surgical instrument members.

20. The method according to claim 19, wherein the surgical position regulators are fixed after the plurality of surgical instrument members is moved to the first surgical region.

21. The method according to claim 19, conically moving the surgical workers about the single port via length and orientation variation.
